(19) United States
(12) Patent Application Publication
(10) Pub. No.: US 2025/0235393 A1
(43) Pub. Date: Jul. 24, 2025

(54) Staged Formula Amniotic Fluid and Method Thereof

(71) Applicant: Zhongtang Wang, Xiamen (CN)

(72) Inventor: Zhongtang Wang, Xiamen (CN)

(21) Appl. No.: 19/032,657

(22) Filed: Jan. 21, 2025

(30) Foreign Application Priority Data
Jan. 24, 2024 (CN) 202410100746.3
Jan. 24, 2024 (CN) 202410100781.5

(51) Int. Cl. A61K 8/64; A61Q 19/00

(52) U.S. Cl. CPC A61K 8/64; A61Q 19/00; A61K 2800/5922

(57) ABSTRACT

A staged formula amniotic fluid includes a primary stage formula amniotic fluid, a secondary stage formula amniotic fluid and a tertiary stage formula amniotic fluid, which can be artificially prepared and configured to provide a suitable microenvironment for tissue repair and maintenance, and replace amniotic fluid within the uterus. The primary stage formula amniotic fluid contains a higher concentration of nutrients such as proteins, glucose, phospholipids and supplements being used to promote tissue repair, the secondary stage formula amniotic fluid contains a decreased amount of nutrients being used to reinforce the effects of tissue repair, and the tertiary stage formula amniotic fluid contains no nutrients or only a small quantity being used for tissue maintenance.

### CROSS REFERENCE OF RELATED APPLICATION

This application is a non-provisional application that claims the benefit of priority under 35 U.S.C. § 119 to a Chinese application, application Ser. No. 202410100746.3, filed Jan. 24, 2024, and a Chinese application, application Ser. No. 202410100781.5, filed Jan. 24, 2024, which are incorporated herewith by references in their entireties.

### BACKGROUND OF THE PRESENT INVENTION

### Field of Invention

The present invention relates to a field of tissue injury repair and skin cosmetology, more particularly to a formula, usage and application of a staged formula amniotic fluid and a method thereof.

### Description of Related Arts

In nature, tissue injury repair relies on the proliferation and differentiation of local stem cells as well as the reprogramming of mature cells, and there is a phenomenon of “scarless healing in fetuses” in mammals. By learning from the laws of nature, if a microenvironment similar to that of fetuses is provided for the wounds of adult individuals, it would surely promote the activation and proliferation of local stem cells and/or the reprogramming of mature cells at the wound site, and then achieve the goal of high-quality physiological and functional repair of the wound.

It has been detected that the composition of amniotic fluid changes with the progress of pregnancy. In the early stage of pregnancy, the amniotic fluid is mainly secreted by the maternal placenta, which is rich in nutrients and can participate in incubating the embryo at the stage when the skin has not yet formed. While in the late stages of pregnancy, when the fetal skin has formed, the amniotic fluid is mainly composed of fetal urine, with a significant reduction in nutrients, which can participate in fetal protection and skin care.

At present, the process uses normal saline or Ringer's solution to replace the amniotic fluid. Because these solutions do not contain the necessary nutrients, they might affect the normal growth and development of the fetus.

In addition, different stages of tissue repair or skin care require different local microenvironments. However, the existing preparations for tissue repair or skin care have fixed active ingredients, neither providing corresponding repair microenvironments according to the different stages of tissue repair, nor effectively promoting the proliferation of local stem cells and/or the reprogramming of mature cells. Therefore, it is difficult to achieve a high-quality physiological and functional repair.

### SUMMARY OF THE PRESENT INVENTION

The invention is advantageous in that it provides a staged formula amniotic fluid for meeting the requirements of the tissue repair microenvironment which can be artificially prepared.

According to the present invention, the foregoing and other objects and advantages are attained by a staged formula amniotic fluid which comprises a primary stage formula amniotic fluid and a secondary stage formula amniotic fluid.

The primary stage formula amniotic fluid contains a higher concentration of nutrients being used to promote tissue repair, while the secondary stage formula amniotic fluid contains a lower concentration of nutrients being used to consolidate tissue repair.

In one embodiment, the nutrients comprises one or a plurality of combination of a protein, a glucose and a lipid.

In one embodiment, the nutrients comprises a supplement being used to meet the requirements of tissue repair.

In one embodiment, the supplement comprises one or a plurality of combination of an electrolyte, a vitamin, an antibiotic, an analgesic, a hemostatic agent, a hyaluronic acid, a collagen, a gelatin, a tranexamic acid, an enzyme, an exosome, a nanoparticle, a growth factor, a cytokine and/or a hormone.

In one embodiment, the supplement comprises a stem cell being used to accelerate tissue repair process.

In one embodiment, the staged formula amniotic fluid comprises an acid-base buffer being used to dissolve the nutrients and supplements and can provide a suitable pH microenvironment according to the requirements of tissue repair.

In one embodiment, the suitable pH microenvironment ranges from pH 2.5 to 7.4, preferably from pH 4.0 to 6.0.

In one embodiment, the protein content of the primary stage formula amniotic fluid ranges from 20 g/L to 80 g/L, preferably from 20 g/L to 40 g/L.

In one embodiment, the protein content of the second stage formula amniotic fluid ranges from 2 g/L to 20 g/L, preferably from 5 g/L to 10 g/L.

In one embodiment, the glucose content of the primary stage formula amniotic fluid i ranges from 3 mmol/L to 6 mmol/L, preferably from 2 mmol/L to 4 mmol/L.

In one embodiment, the glucose content of the second stage formula amniotic fluid ranges from 1 mmol/L to 3 mmol/L, preferably from 1.5 mmol/L to 2.5 mmol/L.

In one embodiment, the lipids comprise a fatty acids at a concentration from 0 mmol/L to 4 mmol/L, preferably from 0.5 mmol/L to 2 mmol/L, a phospholipids at a concentration from 10 mg/L to 100 mg/L, preferably from 20 mg/L to 50 mg/L, a cholesterol at a concentration from 0 mmol/L to 4 mmol/L, preferably from 0.5 mmol/L to 2.5 mmol/L, and a triglycerides at a concentration from 0 μmol/L to 100 μmol/L, preferably from 5 μmol/L to 20 μmol/L.

In one embodiment, the staged formula amniotic fluid comprises the primary stage formula amniotic fluid and the secondary stage formula amniotic fluid. The primary stage formula amniotic fluid can be prepared in a dust-free and sterile environment by adding one or a plurality of several high-concentration nutrients such as proteins, glucose and lipids, as well as an appropriate amount of supplements, for example, one or a plurality of an electrolyte, a vitamin, an antibiotic, an analgesic, a hemostatic agent, a hyaluronic acid, a collagen, a gelatin, a tranexamic acid, an enzyme, an exosome, a nanoparticle, a growth factor, a cytokine and/or a hormone into a buffer solution, followed by stirring, dissolving, adjusting the pH to a range from 2.5 to 7.4, filtering for sterilization, aseptic packaging, and storing at 2° C. to 8° C. The secondary stage formula amniotic fluid contains lower concentration nutrients, with a preparation process same as that of the primary stage formula amniotic fluid.

In accordance with another aspect of the invention, the present invention provides a staged formula amniotic fluid which comprises a primary stage formula amniotic fluid, a secondary stage formula amniotic fluid and a tertiary stage formula amniotic fluid, which can be artificially prepared. The primary stage formula amniotic fluid contains a higher concentration of nutrients being used to promote the repair of tissue wounds. The secondary stage formula amniotic fluid contains a lower concentration of nutrients being used to consolidate the repair of tissue wounds. The tertiary stage formula amniotic fluid contains no or only a small amount of nutrients being used for tissue care.

In one embodiment, the tertiary stage formula amniotic fluid comprises one or a plurality of combination of a buffer solution, a protein, a glucose, a lipid, a vitamin, an electrolyte, an antibiotic, a nanoparticle, an exosome, a gelatin, a collagen, a hyaluronic acid, a tranexamic acid, a botulinum toxin, a growth factor and/or a hormone.

In one embodiment, the preparation process of the tertiary stage formula amniotic fluid is the same as that of the primary stage formula amniotic fluid.

On the other hand, in one embodiment, a usage of a staged formula amniotic fluid comprises covering surface tissue. The covering surface tissue includes but is not limited to smearing, infiltrating, wet dressing and/or spraying. The surface tissue includes but is not limited to facial and neck skin, scalp, limb skin, hand and foot skin, trunk skin, breast skin, perineum skin, mucous membranes, conjunctiva and/or cornea.

In one embodiment, a usage of a staged formula amniotic fluid comprises perfusing deep tissue. The perfusing deep tissue includes but is not limited to continuous or intermittent flushing, continuous or intermittent infiltration and/or continuous or intermittent bathing. The deep tissue includes but is not limited to oral cavity, nasal cavity, external auditory canal, cranial cavity, thoracic cavity, abdominal cavity, pelvic cavity, digestive tract, bladder, urinary tract, vagina, uterus and/or traumatic cavities.

In one embodiment, a usage of a staged formula amniotic fluid comprises repairing surface tissue injury. The surface tissue includes but is not limited to facial and neck skin, scalp, limb skin, hand and foot skin, trunk skin, breast skin, perineum skin, mucous membranes, conjunctiva and/or cornea. The injury includes trauma, burns, scalds, ulcers, erosions, infections, inflammations, edema, allergies and/or tumors.

In one embodiment, a usage of a staged formula amniotic fluid comprises caring surface tissue. The surface tissue includes but is not limited to facial and neck skin, scalp, limb skin, hand and foot skin, trunk skin, breast skin, perineum skin, mucous membranes, conjunctiva and/or cornea. The caring surface tissue includes but is not limited to hydrating the skin, supplementing collagen to the skin, supplementing hyaluronic acid to the skin, supplementing vitamins to the skin, reducing oil secretion of the skin, shrinking skin pores, fading skin spots, removing wrinkles from the skin, and/or promoting hair growth.

In one embodiment, a usage of a staged formula amniotic fluid comprises repairing deep tissue injury. The deep tissue includes but is not limited to an oral cavity, nasal cavity, external auditory canal, cranial cavity, thoracic cavity, abdominal cavity, pelvic cavity, digestive tract, bladder, urinary tract, vagina, uterus and/or traumatic cavities. The deep tissue injury includes but is not limited to partial organ defects, partial tissue defects, infections, inflammations, bleeding, ulcers, erosions, pain and/or tumors.

In one embodiment, a usage of a staged formula amniotic fluid comprises supplementing amniotic fluid. Preferably, the primary stage formula amniotic fluid is supplemented in the early stage of pregnancy, the secondary stage formula amniotic fluid is supplemented in the middle stage of pregnancy, and the tertiary stage formula amniotic fluid is supplemented in the late stage of pregnancy respectively.

In one embodiment, a usage of a staged formula amniotic fluid comprises replacing amniotic fluid. Preferably, the primary stage formula amniotic fluid is chosen in the early stage of pregnancy, the secondary stage formula amniotic fluid is chosen in the middle stage of pregnancy, and the tertiary stage formula amniotic fluid is chosen in the late stage of pregnancy.

Beneficial Effects:
1. The staged formula amniotic fluid of the present application can simulate the nutrient components of amniotic fluid in different stages of pregnancy. Based on the basic components, the supplement can be increased or decreased according to a specific situation. For example, a formula for infected tissues can have an increased amount of antibiotics and/or a decreased pH, a formula for edematous tissues can have an increased osmotic pressure and/or added hormones, a formula for aged tissues can have added growth factors and/or increased nutrient components, a formula for tissues with ulcer bleeding can have added hemostatic agents, a formula for painful tissues can have added analgesics, a formula for tissues with excessive sebum secretion can contain no fats, a formula for dry skin can have an increased fat content, a formula for normal skin can have added transdermal agents, and a formula for skin with pigmentation can have added agents for fading spots. Thus, the staged formula amniotic fluid can meet the specific needs of various tissue repairs and skin care, and improve the curative effect.
2. The staged formula amniotic fluid of the present application can simulate the nutrient components in different stages of pregnancy, which can be used for amniotic fluid supplementation or replacement treatments in different stages of pregnancy, providing a better intra-amniotic microenvironment and being conducive to the normal growth and development of the fetus.
3. According to the repair characteristics and types of surface tissues and deep tissues, a plurality of universal types of staged formula amniotic fluid can be constructed in the embodiments of the present application, which can reduce the preparation cost of the staged formula amniotic fluid and increase economic benefits.

These and other objectives, features, and advantages of the present invention will become apparent from the following detailed description, the accompanying drawings, and the appended claims.

### BRIEF DESCRIPTION OF THE DRAWINGS

In order to illustrate the technical solutions of the embodiments of the present application more clearly, the following will briefly introduce the accompanying drawings that need to be used in the description of the embodiments. Obviously, the accompanying drawings in the following description are only some embodiments of the present application. For those skilled in the art, other accompanying drawings can also be obtained based on these accompanying drawings without creative efforts.

In addition, the accompanying drawings are only schematic illustrations of the present application and are not necessarily drawn to scale. The same reference numerals in the figures denote the same or similar parts, so the repeated descriptions of them will be omitted. Some block diagrams shown in the accompanying drawings are functional entities, which do not necessarily correspond to physically or logically independent entities. These functional entities can be implemented in one or a plurality of a hardware module or a component.

FIG. 1 is a schematic view of a front-view structure of a staged formula amniotic fluid microneedle facia mask applied to a face according to a preferred embodiment of the present application.

FIG. 2 is a schematic sectional view illustrating a sectional-view structure of a staged formula amniotic fluid microneedle facial mask according to the preferred embodiment of the present application.

FIG. 3 is a schematic sectional view illustrating a sectional-view structure of a solid microneedle according to the preferred embodiment of the present application.

FIG. 4 is a schematic sectional view illustrating a sectional-view structure of a hollow microneedle according to the preferred embodiment of the present application.

FIG. 5 is a schematic sectional view illustrating a sectional-view structure of a grooved microneedle according to the preferred embodiment of the present application.

FIG. 6 is a schematic view illustrating an arrangement structure of a solid microneedle matrix of a staged formula amniotic fluid microneedle facial mask according to the preferred embodiment of the present application.

FIG. 7 is a schematic view illustrating an arrangement structure of a hollow microneedle matrix of a staged formula amniotic fluid microneedle facial mask according to the preferred embodiment of the present application.

FIG. 8 is a schematic view illustrating an arrangement structure of a hollow microneedle matrix of another staged formula amniotic fluid microneedle facial mask according to the preferred embodiment of the present application.

FIG. 9 is a schematic view illustrating a structural state of a staged formula amniotic fluid microneedle facial mask before pressing according to the preferred embodiment of the present application.

FIG. 10 is a schematic view illustrating a structural state of a staged formula amniotic fluid microneedle facial mask during pressing according to the preferred embodiment of the present application.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

In the description of the present invention, unless explicitly stated otherwise and qualified, degree terms such as “increase”, “decrease”, “addition”, “high”, and “low” and the directional terms such as “surface” or “deep” are only relative concepts to each other and should be construed broadly.

### Staged Formula Amniotic Fluid Composition

### The Staged Formula Amniotic Fluid Composition Formula 1

According to one preferred embodiment of the present invention, a staged formula amniotic fluid is provided, which can be used to meet the needs of tissue repair microenvironment. Referring to the parameters such as the composition, pH, and osmotic pressure of amniotic fluid in the early, middle, and late stages of human pregnancy, the staged formula amniotic fluid can be artificially prepared.

According to the different nutrient component content, the staged formula amniotic fluid in the preferred embodiment of the present application comprises a primary stage formula amniotic fluid and a secondary stage formula amniotic fluid. The primary stage formula amniotic fluid contains a relatively higher concentration of nutrient components and is used to promote tissue repair, while the secondary stage formula amniotic fluid contains a lower concentration of nutrient components and is used to consolidate tissue repair.

It should be noted that the nutrient components in the preferred embodiment of the present application might comprise one or more combinations of a plurality of proteins, glucose, and lipids, which are used to meet the basic material requirements for tissue repair. Among them, the proteins include but are not limited to albumin, globulin, polypeptides, amino acids, collagen and/or fibroin. The lipids include but are not limited to fatty acids, phospholipids, cholesterol and/or triglycerides.

The nutrient components in the preferred embodiment of the present application can further comprise a supplement to meet specific requirements for tissue repair. For example, a formula for infected tissues may have an increased amount of antibiotics and/or a decreased pH, a formula for edematous tissues may have an increased osmotic pressure and/or added hormones, a formula for aged tissues may have added growth factors and/or increased nutrient components, a formula for tissues with ulcer bleeding may have added hemostatic agents, a formula for painful tissues may have added analgesics, a formula for tissues with excessive sebum secretion may have reduced or no lipids, a formula for dry skin may have an increased lipid content, a formula for normal skin may have added transdermal agents, a formula for skin with pigmentation may have added agents for fading spots such as tranexamic acid and/or coenzyme and/or hydroquinone, a formula for supplementing skin moisture may have added hyaluronic acid, a formula for increasing skin thickness and reducing sagging may have added collagen, a formula for reducing inflammation may have added cytokines that inhibit inflammation, and a formula for improving skin quality may have added exosomes and/or nanoparticles. Thus, the staged formula amniotic fluid can meet the specific needs of various tissue repairs and skin care, and improve the curative effect.

In addition, a stabilizer is also an essential component, which is used to maintain the activity of the nutrient components and supplements and prevent the staged formula amniotic fluid from denaturing and/or deteriorating.

Generally, the supplement of the staged formula amniotic fluid in the preferred embodiment of the present application may comprise one or a plurality of combination of an electrolyte, a vitamin, an antibiotic, an analgesic, a hemostatic agent, a hyaluronic acid, a collagen, a gelatin, a tranexamic acid, an enzyme, a hormone, an exosome, a nanoparticle, a growth factor and/or a cytokine. The supplements such as antibiotics, analgesics, hemostatic agents, tranexamic acid, hormones, growth factors and/or cytokines can be added to the staged formula amniotic fluid accordance with the usage and dosage specified in current laws, regulations, and/or industry standards, and are used strictly under the guidance of doctors.

In addition, the electrolytes can be used to maintain a local osmotic pressure. An iso-osmotic staged formula amniotic fluid is mainly used, and hyperosmotic or hypoosmotic staged formula amniotic fluid can also be prepared.

Moreover, the supplement in the preferred embodiment of the present application can also comprise a stem cell, which is used to accelerate tissue repair. The stem cell can be obtained from the user's own tissues or from a stem cell bank. The content of stem cells ranges from 1×10³/L to 1×10⁷/L, preferably from 1×10⁴/L to 1×10⁶/L.

It can be understood that with a progress of science and technology and a deeper understanding of tissue repair and skin care, a new supplement suitable for being added to the staged formula amniotic fluid will continue to emerge.

In addition, the staged formula amniotic fluid in the preferred embodiment of the present application can comprise an acid-base buffer, such as carbonate buffer and phosphate buffer, which is used to dissolve nutrient components and supplements and can also provide a suitable and stable pH microenvironment according to the needs of tissue repair. Generally, the suitable pH microenvironment for tissue repair is from pH 2.5 to pH 7.0, preferably from pH 4.0 to pH 6.0. The suitable pH microenvironment for skin care is from pH 3.5 to pH 6.0, preferably from pH 4.5 to pH 5.5. Additionally, a specific site may require a specific pH microenvironment. For example, the suitable pH microenvironment for vaginal mucosa is from pH 2.5 to pH 3.5, and the suitable pH microenvironment for infected site is from pH 3.5 to pH 4.5.

It should be noted that the higher or lower concentration of the nutrient components in the staged formula amniotic fluid in the preferred embodiment of the present application is relative. Generally, the primary stage formula amniotic fluid contains a relatively higher concentration of nutrient components, which can provide sufficient repair materials for damaged tissues. The protein content in the primary stage formula amniotic fluid is from 20 g/L to 80 g/L, preferably from 30 g/L to 50 g/L. According to actual needs, the protein content in the primary stage formula amniotic fluid can also be formulated from 20 g/L to 30 g/L, from 3 g/L to 40 g/L, from 41 g/L to 50 g/L, from 51 g/L to 60 g/L, from 61 g/L to 70 g/L or from 71 g/L to 80 g/L. Among them, the proportion of albumin is generally not less than 50%. The glucose content in the primary stage formula amniotic fluid is from 3 mmol/L to 6 mmol/L, preferably from 2 mmol/L to 4 mmol/L. According to actual needs, the glucose content in the primary amniotic fluid can also be formulated from 3 mmol/L to 4 mmol/L, from 4 mmol/L to 5 mmol/L or from 4 mmol/L to 6 mmol/L.

In addition, the staged formula amniotic fluid in the preferred embodiment of the present application can comprise a fatty acid at a concentration from 0 mmol/L to 4 mmol/L, preferably from 0.5 mmol/L to 2 mmol/L, a phospholipids at a concentration from 10 mg/L to 100 mg/L, preferably from 20 mg/L to 50 mg/L, a cholesterol at a concentration from 0 mmol/L to 4 mmol/L, preferably from 0.5 mmol/L to 2.5 mmol/L, and/or a triglyceride at a concentration from 0 μmol/L to 100 μmol/L, preferably from 5 μmol/L to 20 μmol/L. Generally, the lipids content in the primary stage formula amniotic fluid can be slightly higher than that in the secondary stage formula amniotic fluid, or they may be the same. It should be noted that when it is necessary to increase the transdermal absorption of fat-soluble vitamins, such as vitamin E and vitamin D, the lipids content in the staged formula amniotic fluid can be increased. When it is necessary to increase the transdermal absorption of water-soluble vitamins, such as vitamin C, the lipids content in the staged formula amniotic fluid can be reduced.

The preparation of the primary stage formula amniotic fluid in an embodiment of the present application can be carried out in a dust-free and sterile environment, such as a GMP workshop, a sterile operating room, a sterile glove box, or a super clean bench. The preparation of the primary stage formula amniotic fluid can comprise steps of:
(1) preparing various nutrient components of the primary stage formula amniotic fluid, such as proteins, glucose, lipids, and so on;
(2) preparing a supplement, such as electrolytes, vitamins, antibiotics, analgesics, hemostatic agents, hyaluronic acid, collagen, gelatin, tranexamic acid, enzymes, exosomes, nanoparticles, growth factors, cytokines, hormones, and so on;
(3) preparing a buffer;
(4) adding a combination of one or more of high-concentration nutrient components, such as proteins, glucose, and lipids, and an appropriate amount of supplement, such as a combination of electrolytes, vitamins, antibiotics, analgesics, hemostatic agents, hyaluronic acid, collagen, gelatin, tranexamic acid, enzymes, exosomes, nanoparticles, growth factors, cytokines and/or hormones, into the buffer;
(5) stirring and dissolving them to obtain the primary stage formula amniotic fluid base solution with a protein concentration from 20 g/L to 80 g/L, a glucose concentration from 3 mmol/L to 6 mmol/L, a fatty acid concentration from 0.5 mmol/L to 2 mmol/L, a phospholipid concentration from 20 mg/L to 50 mg/L, a cholesterol concentration from 0.5 mmol/L to 2.5 mmol/L, a triglyceride concentration from 5 μmol/L to 20 μmol/L, a sodium chloride from 5 g/L to 8 g/L of, a potassium chloride from 0.2 g/L to 0.5 g/L, and a calcium chloride from 0.2 g/L to 0.5 g/L;
(6) adjusting the pH with hydrochloric acid or sodium hydroxide so that the pH is between pH 2.5 and pH 7.4;
(7) filtering for sterilization;
(8) packaging aseptically; and
(9) obtaining the primary stage formula amniotic fluid, which is stored at 2° C. to 8° C.

It should be noted that the supplements such as vitamins, antibiotics, analgesics, hemostatic agents, hyaluronic acid, collagen, gelatin, tranexamic acid, enzymes, exosomes, nanoparticles, growth factors, cytokines and/or hormones can be re-formulated when using the primary stage formula amniotic fluid. The supplements are added to the primary stage formula amniotic fluid as needed to achieve a better therapeutic and/or maintenance effect.

It can be understood that one unit of the primary stage formula amniotic fluid base powder including protein from 20 g to 80 g, glucose from 0.5 g to 1.0 g, fatty acid from 0.2 g to 0.5 g, phospholipid from 20 mg to 50 mg, cholesterol from 0.2 g to 0.5 g, triglyceride from 0.004 g to 0.02 g, sodium chloride from 5 g to 8 g, potassium chloride from 0.2 g to 0.5 g, calcium chloride from 0.2 g to 0.5 g, sodium lactate from 0.1 g to 3.0 g, sodium dihydrogen phosphate from 0.2 g to 2.0 g, and/or potassium dihydrogen phosphate from 0.1 g to 0.3 g can be prepared according to a proportion. When using, one unit of the primary stage formula amniotic fluid base powder can be dissolved into 1L of pure water to obtain the primary stage formula amniotic fluid. Then the pH can be adjusted between 2.5 and 7.4 as needed, and the supplements can be added to the primary stage formula amniotic fluid to achieve a better therapeutic or maintenance effect.

### The Staged Formula Amniotic Fluid Composition Formula 2

According to one preferred embodiment of the present application, the secondary stage formula amniotic fluid can comprise a protein with a content from 2 g/L to 20 g/L, preferably from 5 g/L to 10 g/L. According to actual needs, the protein content in the secondary stage formula amniotic fluid can also be formulated as 2 g/L to 5 g/L, 6 g/L to 10 g/L, 11 g/L to 15 g/L or 16 g/L to 20 g/L. Among them, the proportion of albumin is generally not less than 50%. The secondary stage formula amniotic fluid can comprise a glucose with a content from 1 mmol/L to 3 mmol/L, preferably from 1.5 mmol/L to 2.5 mmol/L. It can be understood that the secondary stage formula amniotic fluid used by diabetic patients may contain no glucose. In addition, the secondary stage formula amniotic fluid for rinsing and/or infiltrating infected tissues may contain no glucose.

The preparation of the secondary stage formula amniotic fluid in the preferred embodiment of the present application can be carried out in a dust-free and sterile environment, such as a GMP workshop, a sterile operating room, a sterile glove box, or a super clean bench. The preparation of the secondary stage formula amniotic fluid can comprise steps of:
(21) preparing various nutrient components of the secondary stage formula amniotic fluid, such as proteins, glucose, lipids, and so on;
(22) preparing a supplement, such as electrolytes, vitamins, antibiotics, analgesics, hemostatic agents, hyaluronic acid, collagen, gelatin, tranexamic acid, enzymes, exosomes, nanoparticles, growth factors, cytokines, hormones, and so on;
(23) preparing a buffer;
(24) adding a combination of one or several of low-concentration nutrient components, such as proteins, glucose, and lipids, and an appropriate amount of supplement, such as a combination of one or several of electrolytes, vitamins, antibiotics, analgesics, hemostatic agents, hyaluronic acid, collagen, gelatin, tranexamic acid, enzymes, exosomes, nanoparticles, growth factors, cytokines, and hormones, into the buffer;
(25) stirring and dissolving them to obtain the secondary stage formula amniotic fluid base solution with a protein concentration from 2 g/L to 20 g/L, a glucose concentration from 1 mmol/L to 3 mmol/L, a fatty acid concentration from 0.5 mmol/L to 2 mmol/L, a phospholipid concentration from 20 mg/L to 50 mg/L, a cholesterol concentration from 0.5 mmol/L to 2.5 mmol/L, a triglyceride concentration from 5 μmol/L to 20 μmol/L, a sodium chloride from 5 g/L to 8 g/L of, a potassium chloride from 0.2 g/L to 0.5 g/L, and a calcium chloride from 0.2 g/L to 0.5 g/L;
(26) adjusting the pH with hydrochloric acid or sodium hydroxide so that the pH is between 2.5 and 7.4;
(27) filtering for sterilization;
(28) packaging aseptically; and
(29) obtaining the secondary stage formula amniotic fluid, which is stored at 2° C. to 8° C.

It should be noted that the supplements such as vitamins, antibiotics, analgesics, hemostatic agents, hyaluronic acid, collagen, gelatin, tranexamic acid, enzymes, exosomes, nanoparticles, growth factors, cytokines and/or hormones can be re-formulated when using the secondary stage formula amniotic fluid. The supplements are added to the secondary stage formula amniotic fluid as needed to achieve a better therapeutic and/or maintenance effect.

It can be understood that one unit of the secondary stage formula amniotic fluid base powder including protein from 2 g to 20 g, glucose from 0.1 g to 0.5 g, fatty acid from 0.2 g to 0.5 g, phospholipid from 20 mg to 50 mg, cholesterol from 0.2 g to 0.5 g, triglyceride from 0.004 g to 0.02 g, sodium chloride from 5 g to 8 g, potassium chloride from 0.2 g to 0.5 g, calcium chloride from 0.2 g to 0.5 g, sodium lactate from 0.1 g to 3.0 g, sodium dihydrogen phosphate from 0.2 g to 2.0 g, and/r potassium dihydrogen phosphate from 0.1 g to 0.3 g can be prepared according to a proportion. When using, one unit of the secondary stage formula amniotic fluid base powder can be dissolved into 1L of pure water to obtain the secondary stage formula amniotic fluid. Then the pH can be adjusted between 2.5 and 7.4 as needed, and the supplements can be added to the secondary stage formula amniotic fluid to achieve a better therapeutic or maintenance effect.

In addition, a several general-purpose staged formula amniotic fluids can be developed based on the repair characteristics of surface tissues and deep tissues. Thus, the preparation cost of the staged formula amniotic fluid can be reduced and economic benefits can be improved.

### The Staged Formula Amniotic Fluid Composition Formula 3

According to one preferred embodiment of the present invention, a staged formula amniotic fluid can be used to meet the needs of the tissue repair microenvironment. Referring to the parameters such as the composition, pH, and osmotic pressure of amniotic fluid in the early, middle, and late stages of human pregnancy, the staged formula amniotic fluid is artificially prepared. According to the different nutrient component contents, the staged formula amniotic fluid can comprise a primary stage formula amniotic fluid, a secondary stage formula amniotic fluid, and a tertiary stage formula amniotic fluid. The tertiary stage formula amniotic fluid in an embodiment of the present application contains no or only a slight amount of nutrient and can be widely used for tissue care. For example, for the care of dry skin, it can contain lipids but may not contain proteins and glucose.

The tertiary stage formula amniotic fluid in the preferred embodiment of the present application can comprise a combination of one or a plurality of supplements such as electrolytes, vitamins, antibiotics, nanoparticles, exosomes, gelatin, collagen, hyaluronic acid, tranexamic acid, botulinum toxin, growth factors and/or hormones, in addition to a small amount of protein, glucose and/or lipids.

According to the preferred embodiment of the present application, the tertiary stage formula amniotic fluid comprises a protein with a content from 0 g/L to 2 g/L. According to actual needs, the protein content can also be formulated as 0 g/L to 0.5 g/L, 0.6 g/L to 1.0 g/L or 1.1 g/L to 2.0 g/L. Among them, the proportion of albumin is generally not less than 50%. The tertiary stage formula amniotic fluid comprises a glucose with a content from 0 mmol/L to 3 mmol/L. According to actual needs, the glucose content in the tertiary stage formula amniotic fluid can also be formulated as 0 mmol/L to 1.0 mmol/L, 1.1 mmol/L to 2 mmol/L or 2.1 mmol/L to 3 mmol/L. It can be understood that the tertiary stage formula amniotic fluid used by diabetic patients may contain no glucose. In addition, the tertiary stage formula amniotic fluid for rinsing and/or infiltrating infected tissues may contain no glucose.

In addition, the tertiary stage formula amniotic fluid in the preferred embodiment of the present application may comprise fatty acids with a content from 0 mmol/L to 4 mmol/L, preferably from 0.5 mmol/L to 2 mmol/L, a phospholipid with a content from 10 mg/L to 100 mg/L, preferably from 20 mg/L to 50 mg/L, a cholesterol with a content from f 0 mmol/L to 4 mmol/L, preferably from 0.5 mmol/L to 2.5 mmol/L, and/or a triglyceride with a content from 0 μmol/L to 100 μmol/L, preferably from 5 μmol/L to 20 μmol/L.

Generally, the tertiary stage formula amniotic fluid in the preferred embodiment of the present application is mainly used for skin or tissue care. If it is used for skin with excessive sebum secretion, the formula can contain no lipids. If it is used for dry skin, the lipids content in the formula can be increased.

Specifically, for a formula of the tertiary stage formula amniotic fluid in the preferred embodiment of the present application used for normal skin, a transdermal agent can be added, for a formula used for skin with pigmentation spots, agents for lightening spots such as tranexamic acid and/or coenzymes can be added, for a formula used to supplement skin moisture, hyaluronic acid can be added, for a formula used to increase skin thickness and relieve relaxation, collagen can be added, for a formula used to reduce inflammation and/or allergies, cytokines that inhibit inflammation can be added, and for a formula used to improve skin quality, exosomes and/or nanoparticles can be added, thus meeting the specific needs of various types of skin care.

In addition, the tertiary stage formula amniotic fluid in the preferred embodiment of the present application may further comprise an acid-base buffer, such as carbonate buffer and phosphate buffer used to dissolve nutrient components and supplements and can also provide a suitable and stable pH microenvironment according to the tissue needs. Generally, the suitable pH microenvironment for the tertiary stage formula amniotic fluid used for skin care is from pH 3.5 to pH 6.0, preferably from pH 4.5 to pH 5.5. In addition, a specific site may require a specific pH microenvironment. For example, the suitable pH microenvironment for vaginal mucosa is pH 2.5 to pH 3.5.

In addition, the tertiary stage formula amniotic fluid in the preferred embodiment of the present application may not contain nutrient components such as proteins, glucose, and lipids, but may include a combination of one or several of supplements like buffers, electrolytes, vitamins, antibiotics, nanoparticles, exosomes, gelatin, collagen, hyaluronic acid, tranexamic acid, botulinum toxin, growth factors and/or hormones.

It should be noted that according to the characteristics and types of surface tissues and deep tissues repair, one or several general-purpose staged formula amniotic fluids can be developed. For example, in an early stage after injury-causing laser treatment when more emphasis is placed on skin repair, the primary stage formula amniotic fluid with the addition of analgesics and/or growth factors in the formula can be used. In the middle and late stages, the secondary stage formula amniotic fluid with the addition of vitamins, exosomes, collagen, hyaluronic acid and/or tranexamic acid in the formula can be adopted. This can reduce the preparation cost of the staged formula amniotic fluid and achieve a purpose of increasing economic benefits. In addition, several general-purpose staged formula amniotic fluids for skin care can also be developed according to the skin types to achieve a goal of reducing the preparation cost as well as increasing economic benefits.

It can be understood that the staged formula amniotic fluid in the preferred embodiment of the present application can comprise a quaternary stage formula amniotic fluid, a quinary stage formula amniotic fluid, a senary stage formula amniotic fluid, etc. according to the nutrient components. In addition, the staged formula amniotic fluid in the preferred embodiment of the present application can further comprise a various special-effect functional amniotic fluids according to the types of supplements, such as a repair-type staged formula amniotic fluid, a moisturizing-type staged formula amniotic fluid, a skin-rejuvenating-type staged formula amniotic fluid, a spot-lightening-type staged formula amniotic fluid, an anti-itching-type staged formula amniotic fluid, an oil-controlling-type staged formula amniotic fluid, a desensitizing-type staged formula amniotic fluid, an anti-wrinkle-type staged formula amniotic fluid, and so on.

In addition, the tertiary stage formula amniotic fluid in the preferred embodiment of the present application contains no or only a small amount of nutrient component. For other preparation processes, please refer to the primary stage formula amniotic fluid and the secondary stage formula amniotic fluid.

### EXAMPLES OF THE USAGE OF THE STAGED FORMULA AMNIOTIC FLUID

### Example 1 of the Usage of the Staged Formula Amniotic Fluid

A usage of the staged formula amniotic fluid in the preferred embodiment of the present application is to cover surface tissues with the staged formula amniotic fluid, aiming to achieve an effect of promoting the repair of damaged surface tissues and/or providing a nourishing effect. The staged formula amniotic fluid in an embodiment of the present application can comprise a primary stage formula amniotic fluid, a secondary stage formula amniotic fluid and a tertiary stage formula amniotic fluid, and a supplement.

According to the preferred embodiment of the present application, the surface tissues mainly refer to the superficial tissues of the human body. The surface tissues may include, but are not limited to, the skin of the face and neck, the skin of the head, the skin of the limbs, the skin of the hands and feet, the skin of the trunk, the skin of the breast, the skin of the perineum, mucous membranes, conjunctiva, cornea and/or hair. The staged formula amniotic fluid in an embodiment of the present application can be made into a staged formula amniotic fluid dressing with a different dosage and combination of ingredient contents according to the characteristics of the surface tissues. For example, a staged formula amniotic fluid facial masks or hydrogels for the skin of the face and neck, a staged formula amniotic fluid head-covers, helmets, or hats for the skin of the head, a staged formula amniotic fluid sleeves or cotton pads for the skin of the limbs, a staged formula amniotic fluid gloves or socks for the skin of the hands and feet, a staged formula amniotic fluid gels or drops for mucous membranes, conjunctiva, or cornea. Moreover, a primary stage formula amniotic fluid dressing can be recommended for an early-staged surface tissue injury, a secondary stage formula amniotic fluid dressing can be recommended for a middle-staged surface tissue injury, and a tertiary stage formula amniotic fluid dressing can be recommended for a late-staged surface tissue injury.

It should be noted that in the preferred embodiment of the present application to cover surface tissues with the staged formula amniotic fluid mentioned above may include, but is not limited to, smearing, soaking, wet-dressing, and/or spraying the surface tissues with the staged formula amniotic fluid. For example, one can use a tool to smear the staged formula amniotic fluid onto the surface tissues, and/or use a tool to intermittently and/or continuously soak, wet-dress, and/or spray the staged formula amniotic fluid onto the surface tissues, so as to achieve the effect of promoting the repair or nourishment of the surface tissues.

### Example 2 of the Usage of the Staged Formula Amniotic Fluid

According to the preferred embodiment of the present application, a usage of the staged formula amniotic fluid is to perfuse into a deep tissue, aiming to achieve an effect of promoting the repair of damaged deep tissue. The staged formula amniotic fluid in the present embodiment can comprise a primary stage formula amniotic fluid, a secondary stage formula amniotic fluid and a tertiary stage formula amniotic fluid, and a supplement.

According to the preferred embodiment of the present application, the deep tissue mainly refers to an internal deep tissue of the human body. The deep tissue may include, but are not limited to, the oral cavity, nasal cavity, external auditory canal, cranial cavity, thoracic cavity, abdominal cavity, pelvic cavity, digestive tract, respiratory tract, bladder, urinary tract, vagina, uterus and/or traumatic cavities.

According to the preferred embodiment of the present application, to perfuse into a deep tissue mentioned above includes, but is not limited to, using a tool to continuously and/or intermittently irrigate, and/or continuously or intermittently infiltrate the deep tissues and/or cavities with the staged formula amniotic fluid so as to achieve the effects of promoting the repair of deep tissue, reducing inflammation, and/or nourishing the deep tissue.

It should be noted that in the preferred embodiment of the present application, the staged formula amniotic fluid used for perfusing the deep tissues can be formulated into different dosage forms or components according to the characteristics of the deep tissue. For example, a staged formula amniotic fluid hydrogel with a slow-release function or degradable dressings can be prepared. Moreover, a primary stage formula amniotic fluid gel can be recommended for an early-staged deep tissue injury, a secondary stage formula amniotic fluid gel can be recommended for a middle-staged deep tissue injury, and a tertiary stage formula amniotic fluid gel can be recommended for a late-staged deep tissue injury. Additionally, for deep tissue injuries accompanied by infection, a staged formula amniotic fluid gel with added antibiotics can be utilized, for those accompanied by bleeding, a staged formula amniotic fluid gel with added hemostatic agents can be utilized, and for deep tissue injuries with defects, a staged formula amniotic fluid gel with added stem cells and/or growth factors can be utilized.

### Example 3 of the Usage of the Staged Formula Amniotic Fluid

According to the preferred embodiment of the present application, a usage of the staged formula amniotic fluid is for the repair of surface tissue injuries. The staged formula amniotic fluid in the present embodiment can comprise a primary stage formula amniotic fluid, a secondary stage formula amniotic fluid and a tertiary stage formula amniotic fluid, and a supplement.

It should be noted that the surface tissue mainly refers to the superficial tissues of the human body. The surface tissue may include, but are not limited to, the skin of the face and neck, the skin of the head, the skin of the limbs, the skin of the hands and feet, the skin of the trunk, the skin of the breast, the skin of the perineum, mucous membranes, conjunctiva and/or the cornea.

It should be noted that the surface tissue injuries can include, but are not limited to, traumatic injuries to body surface tissues, burns, scalds, ulcers, erosions, infections, inflammations, edema, allergies and/or tumors of body surface tissues.

Generally, in an early stage of surface tissue injury, for example, from immediately after the injury to one week, the primary stage formula amniotic fluid can be selected to promote the repair of surface tissue injuries. The primary stage formula amniotic fluid can be added with a supplement that promote tissue injuries repair, such as growth factors and/or stem cells. In a middle stage of surface tissue injury, for example, from one to two weeks after the injury, the secondary stage formula amniotic fluid can be chosen to consolidate the repair of surface tissue injuries. After the surface tissue injuries has basically healed, the tertiary stage formula amniotic fluid can be selected to nourish the surface tissue and reduce scar hyperplasia.

### Example 4 of the Usage of the Staged Formula Amniotic Fluid

According to the preferred embodiment of the present application, a usage of the staged formula amniotic fluid is for the maintenance and nourishment of surface tissue. The staged formula amniotic fluid in the present embodiment can comprise a primary stage formula amniotic fluid, a secondary stage formula amniotic fluid and a tertiary stage formula amniotic fluid, and a supplement.

According to the preferred embodiment of the present application, the surface tissue mainly refers to the superficial tissue of the human body. The superficial tissues may include, but are not limited to, the skin of the face and neck, the skin of the head, the skin of the limbs, the skin of the hands and feet, the skin of the trunk, the skin of the breast, the skin of the perineum, the cornea, the conjunctiva and/or the mucous membranes.

According to the preferred embodiment of the present application, the maintenance and nourishment of surface tissue includes, but is not limited to, hydrating the skin, replenishing collagen in the skin, adding hyaluronic acid to the skin, supplementing vitamins in the skin, reducing sebum secretion in the skin, shrinking skin pores, lightening skin spots, reducing wrinkles, promoting hair growth, irrigating mucous membranes, and/or irrigating the cornea or conjunctiva. Based on the purpose of the maintenance and nourishment of surface tissue, a supplement can be added during the preparation of the staged formula amniotic fluid in the present embodiment to obtain different types of staged formula amniotic fluid, such as a moisturizing-type staged formula amniotic fluid, a skin-rejuvenating-type staged formula amniotic fluid, a spot-lightening-type staged formula amniotic fluid, an itch-relieving-type staged formula amniotic fluid, an oil-controlling-type staged formula amniotic fluid, a desensitizing-type staged formula amniotic fluid, a wrinkle-reducing-type staged formula amniotic fluid, an eye-protecting-type staged formula amniotic fluid, a feminine cleansing type staged formula amniotic fluid, and a mouth-rinsing type staged formula amniotic fluid, etc.

Generally, for the maintenance and nourishment of surface tissue, the tertiary stage formula amniotic fluid is the first choice. Since the tertiary stage formula amniotic fluid contains little or no nutritional components, which can reduce the cost. Additionally, according to the physiological characteristics of surface tissues, the content of supplements can be appropriately increased or decreased.

### Example 5 of the usage of the STAGED FORMULA AMNIOTIC FLUID

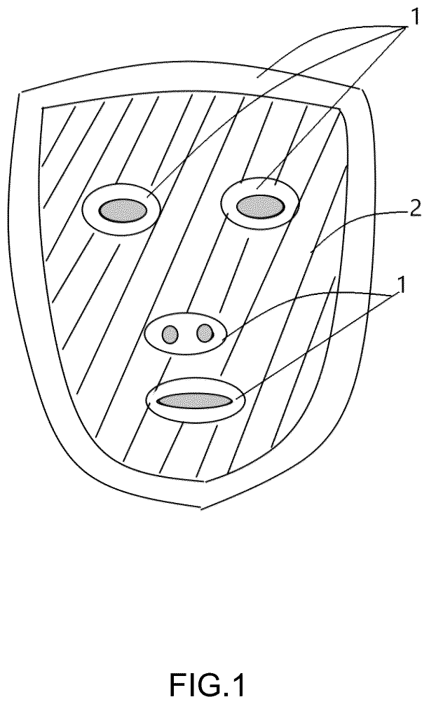

Referring to FIG. 1 to FIG. 10, a staged formula amniotic fluid microneedle facial mask according to one preferred embodiment of the present application comprises a staged formula amniotic fluid (not indicated), a carrier 3, an outer membrane 2, a film 1 and a plurality of microneedles 4 which can create tens of thousands of micro-pores on a body surface and stimulate a proliferation of subcutaneous collagen, while the staged formula amniotic fluid can provide a suitable micro-environment for the repair of the micro-pores.

The staged formula amniotic fluid comprises a primary stage formula amniotic fluid, a secondary stage formula amniotic fluid and a tertiary stage formula amniotic fluid. Generally, the primary stage formula amniotic fluid contains a higher concentration of nutrients configured to promote tissue repair, the secondary stage formula amniotic fluid contains a lower concentration of nutrients configured to consolidate tissue repair, and the tertiary stage formula amniotic fluid contains little or no nutrients configured for tissue maintenance. In addition, a supplement can be added to the staged formula amniotic fluid to meet a specific tissue-repair requirement. Moreover, the staged formula amniotic fluid can comprise a buffer, which can provide a suitable and stable pH micro-environment according to the specific tissue-repair requirement.

As shown in FIG. 1, when the staged formula amniotic fluid microneedle facial mask in an embodiment of the present application is applied to a face, a plurality of openings is reserved for the eyes, nostrils, and mouth. A gap of 5 mm to 10 mm is left between an outer edge of the outer membrane 2 and an upper edge of the neck, an anterior edge of the ear, a hairline edge, and an upper and lower eyelid edges. The film 1 can closely adhere the outer membrane 2 to the skin in the gap, creating a sealed micro-environment on local areas of the face, similar to the micro-environment of a fetus in the mother's uterus.

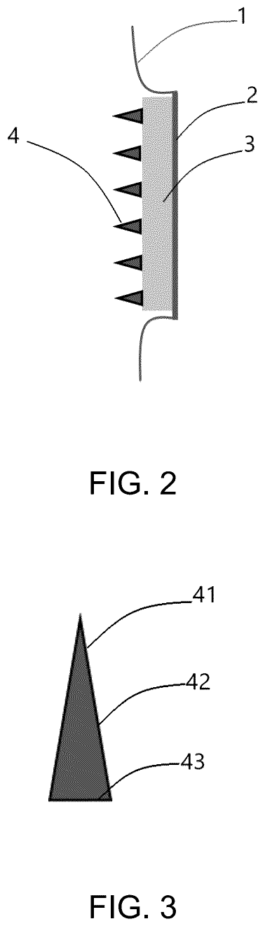

As shown in FIG. 2, the staged formula amniotic fluid microneedle facial mask according to the preferred embodiment of the present application comprises a staged formula amniotic fluid (not indicated), a carrier 3, an outer membrane 2, a film 1 and a plurality of microneedles 4. The carrier 3 can be used to carry and absorb the staged formula amniotic fluid. The staged formula amniotic fluid can be used to provide a micro-environment for skin repair and maintenance. The outer membrane 2 can completely cover the carrier 3 to prevent the staged formula amniotic fluid absorbed by the carrier 3 from evaporating or leaking. The film 1 can stick the outer membrane 2 to a peripheral skin to prevent the staged formula amniotic fluid absorbed by the carrier 3 from leaking, thus creating a sealed micro-environment on the face. The microneedles 4 can be set on an inner side of the carrier 3 and used to create micro-pores on the skin surface, promoting the transdermal absorption of the staged formula amniotic fluid absorbed by the carrier 3.

The carrier 3 can be made of one or several skin-friendly materials such as non-woven fabric, mulberry silk, hydrogel, and/or sponge with a thickness ranging from 0.5 mm to 5 mm, preferably from 1 mm to 3 mm. The outer membrane 2 can be made of a soft water-repellent material, such as PVC plastic, with a thickness ranging from 0.1 mm to 1 mm, preferably from 0.25 mm to 0.5 mm. The film 1 can be made of a soft skin-friendly material, such as PVC plastic with a thickness ranging from 0.1 mm to 1 mm, preferably from 0.25 mm to 0.5 mm, and a width ranging from 5 mm to 30 mm, preferably from 10 mm to 20 mm. In addition, the width of the film 1 used at the lower edge of the outer membrane 2 can even be increased to 50 mm to form a tight and firm adhesion between the outer membrane 2 and the neck skin, preventing loosening and liquid leakage caused by head rotation.

The staged formula amniotic fluid microneedle facial mask according to one preferred embodiment of the present application comprises a primary stage formula amniotic fluid microneedle facial mask, a secondary stage formula amniotic fluid microneedle facial mask and a tertiary stage formula amniotic fluid microneedle facial mask. Additionally, according to the supplement, the staged formula amniotic fluid microneedle facial mask can comprise a plurality of functional stage formula amniotic fluid microneedle facial mask. For example, there are a repair staged formula amniotic fluid microneedle facial mask with added growth factors, a hydrating staged formula amniotic fluid microneedle facial mask with added hyaluronic acid, a skin-rejuvenating staged formula amniotic fluid microneedle facial mask with added collagen, a spot-lightening staged formula amniotic fluid microneedle facial mask with added hydroquinone, an anti-itching staged formula amniotic fluid microneedle facial mask with added peppermint oil, an oil-control staged formula amniotic fluid microneedle facial mask with added surfactants, a desensitizing staged formula amniotic fluid microneedle facial mask with added loratadine, a wrinkle-removing staged formula amniotic fluid microneedle facial mask with added botulinum toxin, and so on.

Referring to FIG. 2 to FIG. 8, the staged formula amniotic fluid microneedle facial mask according to the preferred embodiment of the present application can comprise a staged formula amniotic fluid (not indicated), a carrier 3, an outer membrane 2, a film 1, a plurality of microneedles 4 and a base film 5. The microneedles 4 can be arranged on the base film 5, and the base film 5 can be connected to an inner side of the carrier 3.

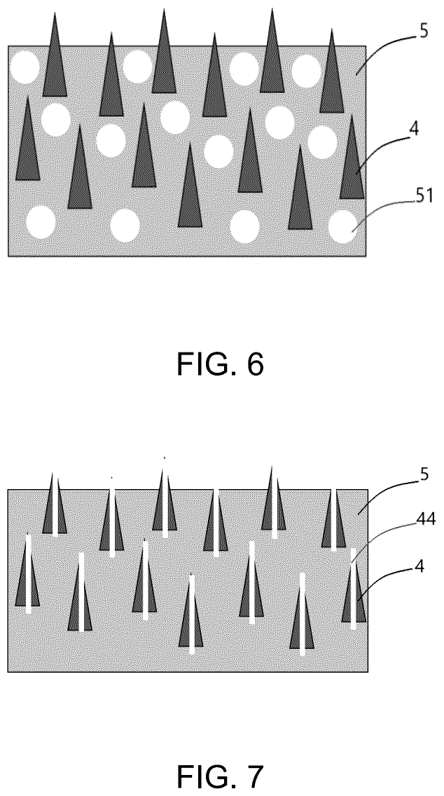

As shown in FIG. 2, FIG. 3 and FIG. 6, a plurality of microneedles 4 of a staged formula amniotic fluid microneedle facial mask in the preferred embodiment of the present application can be arranged in a matrix on the base film 5. The base film 5 can comprise a plurality of holes 51, which can be either scattered and/or arranged in a matrix on the base film 5, and the microneedles 4 and the holes 51 can be distributed alternately. Generally, the microneedles 4 and the base film 5 can be an integrated structure, made of the same material. A needle base 43 of the microneedle 4 can be fixedly connected to the base film 5, and the needle base 43 and the holes 51 can be distributed alternately on the base film 5. When the staged formula amniotic fluid microneedle facial mask is applied to the skin, gently press the outer membrane 2, and indirectly push a needle tip 41 and a needle body 42 of the microneedle 4 to penetrate the skin through the carrier 3. Stop pressing the outer membrane 2, the carrier 3 rebounds, driving the needle tip 41 and the needle body 42 of the microneedle 4 to withdraw from the skin, thus forming micro-channels on the skin surface. The staged formula amniotic fluid and supplement absorbed by the carrier 3 can enter the skin through the holes 51 and the aforementioned micro-channels, achieving the effect of skin repair or maintenance.

In addition, a plurality of microneedles 4 can also be scattered and arranged on the base film 5, with the needle bases 43 and the holes 51 distributed alternately on the base film 5.

The microneedles 4 in the preferred embodiment can be arranged in a matrix on the base film 5. The thickness of the base film 5 can range from 25 μm to 250 μm, preferably from 50 μm to 100 μm. The spacing between the microneedles 4 can be ranging from 200 μm to 5000 μm, preferably from 500 μm to 2000 μm. The holes 51 can be either scattered and/or arranged in a matrix on the base film 5, and an aperture of the holes 51 can be ranging from 50 μm to 5000 μm, preferably from 100 μm to 500 μm.

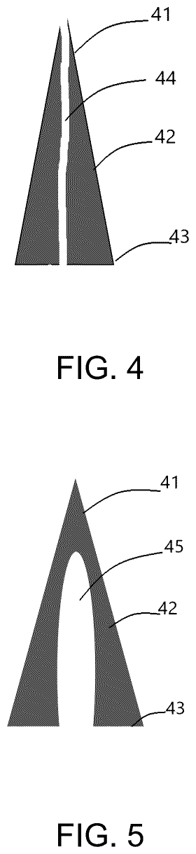

As shown in FIG. 2, FIG. 4 and FIG. 7, the microneedles 4 of a staged formula amniotic fluid microneedle facial mask in the preferred embodiment of the present application can be arranged in a matrix on the base film 5. The microneedles 4 and the base film 5 can be an integrated structure, made of the same material. The needle base 43 of the microneedle 4 can be fixedly connected to the base film 5, and the needle hole 44 of the microneedle 4 can penetrate through the base film 5. When the staged formula amniotic fluid microneedle facial mask is applied to the skin, gently press the outer membrane 2. Through the carrier 3, the needle tip 41 and the needle body 42 of the microneedle 4 can be indirectly pushed to penetrate the skin, and the staged formula amniotic fluid and supplements absorbed by the carrier 3 can be pushed to be injected into the skin through the needle hole 44 of the microneedle 4. After stopping pressing the outer membrane 2, the carrier 3 rebounds, driving the needle tip 41 and the needle body 42 of the microneedle 4 to withdraw from the skin, achieving a micro-injection effect with precise injection levels and dosages.

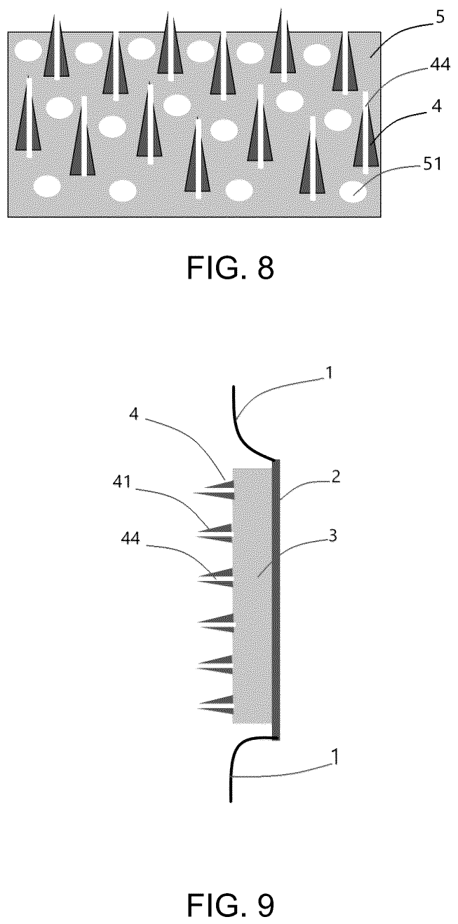

As shown in FIG. 2, FIG. 4 and FIG. 8, the microneedles 4 of a staged formula amniotic fluid microneedle facial mask in the preferred embodiment of the present application can be arranged in a matrix on the base film 5. The base film 5 can incorporate multiple holes 51, which can be either scattered or arranged in a matrix on the base film 5, with the microneedles 4 and the holes 51 distributed in an alternating pattern. The microneedles 4 and the base film 5 can form an integrated structure, crafted from the same material. The needle base 43 of the microneedle 4 can be firmly connected to the base film 5, and the needle hole 44 of the microneedle 4 can run through the base film 5.

When applying staged formula amniotic fluid microneedle facial mask to the skin, gently press the outer membrane 2. This indirectly propels the needle tip 41 and the needle body 42 of the microneedle 4 to pierce the skin via the carrier 3, and simultaneously drives the staged formula amniotic fluid and supplements absorbed by the carrier 3 to be accurately injected into the skin through the needle hole 44 of the microneedle 4. Once the pressure on the outer membrane 2 is released, the carrier 3 rebounds, causing the needle tip 41 and the needle body 42 of the microneedle 4 to retract from the skin, creating micro channels on the skin surface. The staged formula amniotic fluid and supplements absorbed by the carrier 3 can then enter the skin through the holes 51 and the aforementioned micro-channels, achieving an even better skin repair or maintenance effect.

Referring to FIG. 4 and FIG. 7 to FIG. 10, the staged formula amniotic fluid microneedle facial mask according to the preferred embodiment of the present application can comprise a staged formula amniotic fluid (not indicated), a carrier 3, an outer membrane 2, a film 1, a plurality of microneedles 4 and a base film 5. The carrier 3 can absorb the staged formula amniotic fluid and supplements. The microneedles 4 can be set on the base film 5, and the base film 5 can be connected to an inner side of the carrier 3. Each microneedle 4 can comprise a needle tip 41, a needle body 42, a needle base 43 and a needle hole 44. The needle tip 41 can be used to pierce a stratum corneum and a superficial dermis of the skin. The needle base 43 can be used to connect to an inner side of the carrier 3. The needle body 42 can be a main structure of the microneedle 4. One end of the needle hole 44 can run through the base film 5 and then connect to the carrier 3, while the other end can directly reach an interior of the skin when the needle tip 41 pierces the skin. The base film 5 can comprise a plurality of holes 51, which can be either scattered or arranged in a matrix on the base film 5, and the microneedles 4 and the holes 51 can be distributed alternately.

As shown in FIG. 9, when the staged formula amniotic fluid microneedle facial mask is applied to the skin, the microneedles 4 can direct contact with the skin, the film 1 can adhere the outer membrane 2 to the skin around the face, thus creating a sealed space in the local area of the face. The staged formula amniotic fluid and supplements absorbed by the carrier 3 can enter the sealed space, providing a sealed, slightly acidic, low-oxygen-partial-pressure, and moist micro-environment in the local area of the face, which is similar to the micro-environment of a fetus in the mother's uterus.

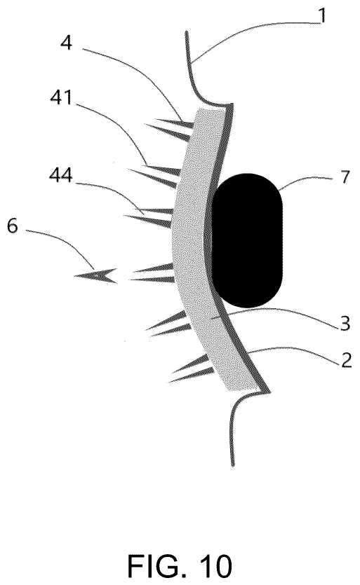

As shown in FIG. 4, FIG. 8 and FIG. 10, when gently patting and pressing the outer membrane 2 with the palm 7, the base film 5 can indirectly pushed through the carrier 3. The base film 5 then propels the needle tip 41 and the needle body 42 of the microneedle 4 to penetrate the skin. Since the area of the outer membrane 2 is much larger than the sum of the cross-sectional area of the needle hole 44 and the area of the hole 51, the pressure from patting generates pressure in the needle hole 44, driving the staged formula amniotic fluid and supplements absorbed by the carrier 3 to form a plurality of liquid mass 6. The liquid masses 6 can be accurately injected into the skin through the needle hole 44 of the microneedle 4. When the pressure on the outer membrane 2 stops, the carrier 3 rebounds, causing the needle tip 41 and the needle body 42 of the microneedle 4 to withdraw from the skin, creating micro-channels on the skin surface. The staged formula amniotic fluid and supplements absorbed by the carrier 3 can then enter the skin through the hole 51 and the aforementioned micro-channels, achieving a better skin repair or maintenance effect.

In addition, as shown in FIG. 4, FIG. 7 and FIG. 10, when gently patting and pressing the outer membrane 2 with the palm 7, the base film 5 is indirectly pushed through the carrier 3. The base film 5 then drives the needle tip 41 and the needle body 42 of the microneedle 4 to penetrate the skin. Given that the area of the outer membrane 2 is far larger than the sum of the cross-sectional areas of the needle holes 44, the pressure from the patting generates pressure, causing the staged formula amniotic fluid and supplements absorbed by the carrier 3 to form a plurality of liquid mass 6. The liquid masses 6 can be accurately injected into the skin through the needle holes 44 of the microneedles 4. When the pressure on the outer membrane 2 ceases, the carrier 3 rebounds, pulling the needle tip 41 and the needle body 42 of the microneedle 4 out of the skin, enabling the precise injection of the staged formula amniotic fluid and supplements absorbed by the carrier 3 into the skin.

It should be understood that the depth of gently patting and pressing the outer membrane 2 with the palm 7 can determine the depth to which the needle tip 41 and the needle body 42 of the microneedle 4 penetrate the skin. The deeper the pressure on the outer membrane 2, the greater the depth to which the needle tip 41 and the needle body 42 of the microneedle 4 penetrate the skin, and the liquid mass 6 can be accurately injected into a deeper layer of the skin through the needle hole 44 of the microneedle 4, yet not exceeding the length of the needle body 42. Additionally, users can pat and press the outer membrane 2 with the palm 7 intermittently or continuously for multiple times, achieving intermittent or continuous and precise injection of the staged formula amniotic fluid and supplements absorbed by the carrier 3 into the skin.

It can be understood that the staged formula amniotic fluid microneedle facial mask in the embodiment is not limited to the repair and/or maintenance of facial skin. It can also be applied to other parts of the body, such as the scalp, neck, back, chest, breasts, abdomen, buttocks, limbs, hands, and/or feet.

### Example 6 of the Usage of the Staged Formula Amniotic Fluid

According to the preferred embodiment of the present application, a usage of the staged formula amniotic fluid is for the repair of deep tissue injuries. The staged formula amniotic fluid in the present embodiment can comprise a primary stage formula amniotic fluid, a secondary stage formula amniotic fluid and a tertiary stage formula amniotic fluid, and a supplement.

It should be noted that the deep tissue mainly refers to the internal deep tissues of the human body. The deep tissues include, but are not limited to, the oral cavity, nasal cavity, cranial cavity, thoracic cavity, abdominal cavity, pelvic cavity, digestive tract, bladder, urinary tract, vagina, uterus and/or traumatic cavities.

It should be noted that the deep tissue injuries include, but are not limited to, partial organ defects, partial tissue defects, infections, inflammations, bleeding, ulcers, erosions, pain and/or tumors.

Specifically, when used for deep tissue injuries, a tool can be used to continuously or intermittently irrigate, and/or continuously or intermittently infiltrate the deep tissues or cavities with the staged formula amniotic fluid, so as to achieve the effects of promoting the repair of deep tissues, reducing inflammation, and/or nourishing the deep tissues. The tool can comprise a wound negative-pressure dressings, a negative-pressure pumps, an infusion pumps, a negative-pressure drainage tube, etc.

In addition, during the early stage of deep tissue injury, for instance, from immediately after the injury to 1 week, the primary stage formula amniotic fluid can be as a priority choice. Moreover, the supplement that can promote tissue repair, such as growth factors and/or stem cells, can be added to the primary stage formula amniotic fluid. If there is a local infection, antibiotics can be added and/or the pH of the primary stage formula amniotic fluid can be lowered. During the middle stage of deep tissue injury, that is, from one to two weeks after the injury, the secondary stage formula amniotic fluid can be chosen to consolidate the repair of the deep tissue injury. After the deep tissue injury has basically healed, the tertiary stage formula amniotic fluid can be selected to nourish the deep tissues, so as to reduce scar hyperplasia and/or tissue adhesion.

Furthermore, when the staged formula amniotic fluid in an embodiment of the present application is used for the repair of deep tissue injuries, forms such as a staged formula amniotic fluid solution, a staged formula amniotic fluid gel, a degradable dressing infiltrated with staged formula amniotic fluid, and an intelligent bionic dressing with staged formula amniotic fluid can be utilized. Through intermittent and/or continuous methods like irrigation, perfusion, wet-dressing, packing, smearing, and/or injection, the staged formula amniotic fluid is applied to the injured sites of deep tissues, thereby achieving the therapeutic effect of promoting the repair of deep tissue injuries.

### Example 7 of the Usage of the Staged Formula Amniotic Fluid

According to the preferred embodiment of the present application, a type of staged formula amniotic fluid can mimic the compositional characteristics of amniotic fluid at different pregnancy stages. By increasing or decreasing supplements based on the basic components, the staged formula amniotic fluid can be used for special wounds or tissues, creating and/or maintaining a local micro-environment that is conducive to the repair of special wounds and/or the nourishment of tissues.

In a formula of a staged formula amniotic fluid in an embodiment of the present application, adding antibiotics and/or lowering the pH can be used to control local tissue infections. A high-osmolarity formula or and/adding hormones to the staged formula amniotic fluid can be used to improve local edematous tissues. Adding growth factors and/or stem cells to the formula can help promote tissue repair and/or anti-aging. Adding hemostatic agents can alleviate wound bleeding, and adding analgesics provides local pain relief. Removing lipids from the formula is beneficial for maintaining skin with excessive sebum secretion, while increasing the lipids content is good for moisturizing dry skin. Adding spot-lightening agents can reduce skin pigmentation. Thus, the formula of the staged formula amniotic fluid can meet the specific needs of various tissue repairs and skin maintenance, improving the therapeutic effect.

In the preferred embodiment of the present application, when the staged formula amniotic fluid is used to create or maintain a local micro-environment conducive to the repair of special wounds or the nourishment of tissues, various forms can be employed, such as a staged formula amniotic fluid solution, a staged formula amniotic fluid gel, a degradable dressing infiltrated with staged formula amniotic fluid, a non-degradable dressing infiltrated with staged formula amniotic fluid, and/or an intelligent bionic dressing with staged formula amniotic fluid. Through intermittent and/or continuous methods like irrigation, perfusion, wet-dressing, packing, smearing and/or injection, the staged formula amniotic fluid can be applied to a special wound or tissue sites of the body, so as to create and/or maintain a local micro-environment that is beneficial to the repair of special wounds or the nourishment of tissues, thereby promoting the repair of special wounds or improving tissue characteristics.

### Example 8 of the Usage of the Staged Formula Amniotic Fluid

According to the preferred embodiment of the present application, a usage of the staged formula amniotic fluid is to replenish the amniotic fluid in the uterus, maintaining a favorable liquid micro-environment required for the growth and development of the fetus. The staged formula amniotic fluid in the present embodiment can comprise a primary stage formula amniotic fluid, a secondary stage formula amniotic fluid and a tertiary stage formula amniotic fluid, and a supplement.

Generally, situations where amniotic fluid supplementation is required may include insufficient amniotic fluid production during pregnancy, amniotic fluid leakage and loss, amniotic fluid loss after amniotic cavity rupture, continuous amniotic fluid loss during fetal surgery, amniotic fluid deficiency after fetal surgery, and/or artificial wombs for incubating premature infants.

When the staged formula amniotic fluid in the preferred embodiments of the present application is used to replenish the amniotic fluid in the uterus, the primary stage formula amniotic fluid can be replenished in the first trimester of pregnancy, the secondary stage formula amniotic fluid in the second trimester, and the tertiary stage formula amniotic fluid in the third trimester. Additionally, when dealing with continuous amniotic fluid loss during fetal surgery, the tertiary stage formula amniotic fluid can be selected for replenishment in the uterus. On one hand, this can meet the basic needs of the fetus, and on the other hand, it can decrease the cost. Moreover, according to the treatment objectives, when used for replenishing amniotic fluid in the uterus, the staged formula amniotic fluid can have a supplement added or reduced. For example, a hemostatic agent can be added to the staged formula amniotic fluid replenished during fetal surgery to reduce bleeding from the surgical wound, and growth factors can be added to the staged formula amniotic fluid replenished after fetal surgery to promote the healing of the fetal incision. It should be noted that when the staged formula amniotic fluid is used to replenish the amniotic fluid in the uterus, the types and dosages of the supplements added to the staged formula amniotic fluid are determined by professional doctors and implemented by professional medical staff to ensure the safety of the mother and the fetus.

When the staged formula amniotic fluid in the preferred embodiment of the present application is used to replenish the amniotic fluid in an artificial womb, the primary stage formula amniotic fluid can be replenished in the early stage of pregnancy, the secondary stage formula amniotic fluid in the middle stage, and the tertiary stage formula amniotic fluid in the late stage. Additionally, compared with the natural maternal uterus, an artificial womb lacks self-secretion and regulation functions. Therefore, the composition combination of the staged formula amniotic fluid and the types of supplements can be adjusted in a timely manner according to the amniotic fluid monitoring indicators within the artificial womb.

It should be noted that when the staged formula amniotic fluid in the embodiments of the application is used for replenishing amniotic fluid in the uterus, the osmotic pressure of the staged formula amniotic fluid ranges from 260 mOsm/L to 320 mOsm/L, with pH between 6.0 and 7.2. Moreover, the injection volume and rate of the staged formula amniotic fluid into the uterus also need to be strictly controlled.

### Example 9 of the Usage of the Staged Formula Amniotic Fluid

According to the preferred embodiment of the present application, a usage of the staged formula amniotic fluid is to replace the amniotic fluid in the uterus, maintaining a favorable liquid micro-environment required for the growth and development of the fetus. The staged formula amniotic fluid in the present embodiment can comprise a primary stage formula amniotic fluid, a secondary stage formula amniotic fluid and a tertiary stage formula amniotic fluid, and a supplement.

Generally, an amniotic fluid contamination, an intra-amniotic cavity infection, an intra-uterine fetal infection, a fetal hemolytic disease, and/or an amniotic fluid contamination in the artificial womb for incubating premature infants can cause the amniotic fluid to become turbid and develop an unpleasant odor. In particular, harmful substances like inflammatory factors and meconium can have an adverse impact on the fetus. In cases mentioned above, replacing the amniotic fluid in the uterus, and/or combined with anti-infection treatment, can improve the fetal living environment to a certain extent and reduce the risk of fetal infection.

When the staged formula amniotic fluid in the embodiments of the application is used to replace the amniotic fluid in the uterus, the amniotic fluid can be replaced with the primary stage formula amniotic fluid in the early pregnancy stage, with the secondary stage formula amniotic fluid in the middle pregnancy stage, and with the tertiary stage formula amniotic fluid in the late pregnancy stage.

In addition, based on the treatment objectives, the staged formula amniotic fluid in the preferred embodiment of the application can have a supplement added or reduced. For instance, in cases of intra-uterine infection, fetal infection, and/or amniotic fluid contamination in the artificial womb for incubating premature infants, antibiotics can be added to the staged formula amniotic fluid according to the results of bacterial culture and drug sensitivity testing to control the infection.

Furthermore, the type and dosage of the staged formula amniotic fluid for replacement are determined by professional doctors and implemented by professional medical staff to ensure the safety of both the mother and the fetus.

It should be noted that when the staged formula amniotic fluid in the preferred embodiment of the application is used to replace the amniotic fluid in the uterus, the osmotic pressure of the staged formula amniotic fluid ranges from 260 mOsm/L to 320 mOsm/L with a pH between 5.0 and 7.2. Moreover, the injection volume and rate of the staged formula amniotic fluid into the uterus need to be strictly controlled. When evidence confirms that an intra-amniotic cavity infection has occurred and/or there is a threat of such an infection, the steps for replacing the amniotic fluid in the uterus may comprise:
S31: withdrawing 90% of the amniotic fluid in the uterus and inject the tertiary stage formula amniotic fluid;
S32: repeating step S31 more than twice;
S33: withdrawing all the amniotic fluid in the uterus; and
S34: injecting the primary stage formula amniotic fluid with added antibiotics in the early stage of pregnancy, the secondary amniotic fluid with added antibiotics in the middle stage of pregnancy, or the tertiary stage formula amniotic fluid with added antibiotics in the late stage of pregnancy.

The staged formula amniotic fluid should maintain a pH between 5.0 and 6.0. The programmed replacement of the amniotic fluid in the uterus above can, on the one hand, achieve the goal of maximizing the purification of the intra-amniotic cavity environment with low-cost, and on the other hand, obtain a synergistic antibacterial therapeutic effect through the combination of antibiotics and an acidified micro-environment.

The above descriptions are merely embodiments of the present application and do not impose any limitations on the technical scope of the application. Therefore, any minor modifications, equivalent changes, and embellishments made to the above-mentioned embodiments based on the technical essence of the application still fall within the scope of the technical solution of the application. Professionals should be aware that, for each specific application, different methods can be used to achieve the described functions, but such implementations should not be regarded as exceeding the scope of the application.

What is claimed is:

1. A primary stage formula amniotic fluid for providing a micro-environment conducive to tissue injury repair, comprising one or more combinations of a protein, a glucose, a lipid, an electrolyte, and a buffer solution.

2. The primary stage formula amniotic fluid according to claim 1, wherein the protein is ranged from 20 g/L to 80 g/L.

3. The primary stage formula amniotic fluid according to claim 1, wherein the protein is ranged from 20 g/L to 40 g/L.

4. The primary stage formula amniotic fluid according to claim 1, wherein the glucose is ranged from 2 mmol/L to 6 mmol/L.

5. The primary stage formula amniotic fluid according to claim 4, wherein the glucose is ranged from 2 mmol/L to 4 mmol/L.

6. The primary stage formula amniotic fluid according to claim 1, wherein the lipid comprising one or more combinations of:
a fatty acid of 0 mmol/L to 4 mmol/L,
a phospholipid of 10 mg/L to 100 mg/L,
a cholesterol of 0 mmol/L to 4 mmol/L, and
a triglyceride of 0 μmol/L to 100 μmmol/L.

7. The primary stage formula amniotic fluid according to claim 6, wherein the lipid comprising one or more combinations of
a fatty acid of 0.5 mmol/L to 2 mmol/L,
a phospholipid of 20 mg/L to 50 mg/L,
a cholesterol of 0.5 mmol/L to 2.5 mmol/L, and
a triglyceride of 5 μmol/L to 20 μmmol/L.

8. The primary stage formula amniotic fluid according to claim 1, further comprising: a supplement which comprises one or more combinations of a vitamin, an antibiotic, an analgesic, a hemostatic, a hyaluronic acid, a collagen, a gelatin, a tranexamic acid, an enzyme, an exosome, a nanoparticle, a growth factor, a cytokine, and a hormone.

9. The primary stage formula amniotic fluid according to claim 1, wherein the buffer solution is configured to provide a suitable pH micro-environment for tissue injury repair.

10. The primary stage formula amniotic fluid according to claim 9, wherein the suitable pH micro-environment is ranged from pH 2.5 to pH 7.2, preferably from pH 4.0 to pH 6.0.

11. A secondary stage formula amniotic fluid for providing a micro-environment conducive to consolidate tissue repair, comprising one or a plurality of combination of a protein, a glucose, a lipid, an electrolyte and a buffer solution.

12. The secondary stage formula amniotic fluid according to claim 11, wherein the protein content ranges from 2 g/L to 20 g/L, preferably from 5 g/L to 10 g/L.

13. The secondary stage formula amniotic fluid according to claim 11, wherein the glucose content ranges from 1 mmol/L to 3 mmol/L.

14. The secondary stage formula amniotic fluid according to claim 11, wherein the lipid comprising:
a fatty acid with a content of 0 mmol/L to 4 mmol/L,
a phospholipid with a content of 10 mg/L to 100 mg/L,
a cholesterol with a content of 0 mmol/L to 4 mmol/L, and/or
a triglyceride with a content of 0 μmol/L to 100 μmmol/L.

15. The secondary stage formula amniotic fluid according to claim 11, wherein the buffer solution is configured to provide a suitable pH micro-environment for consolidating tissue repair, wherein a suitable pH micro-environment is ranged from pH 2.5 to pH 7.2, preferably from pH 4.0 to pH 6.0.

16. The secondary stage formula amniotic fluid according to claim 11, further comprising a supplement which comprises one or more combination of a vitamin, an antibiotic, an analgesic, a hemostatic, a hyaluronic acid, a collagen, a gelatin, a tranexamic acid, an enzyme, an exosome, a nanoparticle, a growth factor, a cytokine, and a hormone.

17. A tertiary stage formula amniotic fluid for providing a micro-environment conducive to tissue maintenance, comprising one or more combinations of a buffer solution, a protein, a lipid, a glucose, a vitamin, an electrolyte, a gelatin, a collagen, a hyaluronic acid, a tranexamic acid, a growth factor, a cytokine, an enzyme, an exosome, and a nanoparticle.

18. The tertiary stage formula amniotic fluid according to claim 17, wherein the protein is ranged from 0 g/L to 2 g/L.

19. The tertiary stage formula amniotic fluid according to claim 17, wherein the lipid comprising one or more combinations of:
a fatty acid of 0 mmol/L to 4 mmol/L,
a phospholipid of 10 mg/L to 100 mg/L,
a cholesterol of 0 mmol/L to 4 mmol/L, and
a triglyceride of 0 μmol/L to 100 μmmol/L.

20. The tertiary stage formula amniotic fluid according to claim 17, wherein the buffer solution is configured to provide a suitable pH micro-environment for tissue maintenance, wherein a suitable pH micro-environment is ranged from pH 2.5 to pH 7.2, preferably from pH 4.0 to pH 6.0.
